(19) United States
(12) Patent Application Publication
(10) Pub. No.: US 2007/0168022 A1
(43) Pub. Date: Jul. 19, 2007

(54) Heart valve

(76) Inventor: Charles J. Eldridge, Burlington, NJ (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/333,957

(22) Filed: Jan. 17, 2006

(51) Int. Cl. A61F 2/24

(52) U.S. Cl. 623/2.4; 623/2.17

(57) ABSTRACT

An embodiment of a valve has a frame defining cusps at a downstream end. Valve leaflets are attached to the frame and arranged to allow fluid flow within the frame in a downstream direction but not in an upstream direction. A sewing ring is provided at the downstream end of the valve. A shroud connects the sewing ring to the frame.

### BACKGROUND

The invention relates generally to valves and especially, but not exclusively, to an artificial valve for replacing the mitral valve in the human heart.

It has been known for many years that the valves of the heart may be defective, or may deteriorate, resulting in a loss of heart function, and various attempts have been made to replace or repair the valves. One form of artificial valve that has been extensively used for replacing the mitral valve of the human heart comprises a wire frame. The frame typically comprises a sewing ring and three cusps or posts extending downstream from the sewing ring. The cusps may be formed by three arcs of wire, the middles of which lie along the sewing ring and the ends of which meet in the cusps. Between the cusps of the frame extend three leaflets of flexible material that spread to close off the opening within the frame in response to fluid flow upstream (in the direction from the cusps towards the sewing ring) and that fold apart to allow fluid flow downstream. The frame is covered with, and the valve leaflets are formed from, physiologically compatible material. The outside of the frame is shrouded with similar material to prevent fluid flow from by-passing the valve leaflets by flowing through the frame. The physiologically compatible material may be autograft (tissue from another part of the patient), homograft (tissue from a human donor other than the patient), heterograft (tissue from a non-human donor, usually porcine or bovine), or synthetic. The polyethylene terephthalate (PET) polyester fiber material sold under the registered trademark DACRON has been widely and successfully used.

Although the above-described conventional heart valve has been widely used with great success, it is not perfect. First, the valve is installed by sewing the sewing ring to the annulus between the atrium and the left ventricle. However, the annulus is in many patients at least partly calcified. Sewing through the calcification is impractical. Removing the calcification may weaken the tissue structure around the annulus unacceptably, leading to other complications, potentially including rupture of the atrio-ventricular groove, and may release harmful debris into the patient's bloodstream.

Second, because the conventional heart valve extends downstream from the sewing ring, the cusps of the frame project into the left ventricle. There is consequently a risk of perforation or other damage to the wall of the ventricle from the artificial valve.

Various attempts have been made to install a replacement mitral valve intra-atrially. DeLeon et al., Ann. Thorac. Surg. 1999; 68:1843-5, describe inserting a porcine heterograft valve in a conduit upstream of the intact but defective mitral valve. The distal (downstream) end of the conduit was sutured to the mitral annulus, and the proximal end, with the base of the porcine valve, was sutured to the atrial wall. This resulted in a dead space between the conduit and the atrial wall, which was drained into the right atrium through a hole in the atrial septum.

Sir Donald Ross, FRCS, has developed a technique for autograft from the pulmonary valve to the mitral valve site, known as the “Ross II” technique. The Ross II technique involves constructing a tube of autograft material and PET fiber fabric, typically of a PET fabric layer covered in two layers of pericardium tissue, with its distal end sutured to the mitral annulus. The flexibility of the tube has caused problems, and Ross therefore prefers to construct a pericardial collar forming a false atrial floor from the proximal end of the valve to the wall of the atrium. Because of the combined shape of the tube and collar, this has become known as the “top hat” technique. Kabbani et al., Ann. Thorac. Surg. 2001; 72: 947-950, have proposed omitting the collar forming the false atrial floor, in order to connect the dead space to the atrium. Kabbani's technique requires a very stiff PET fabric tube to support the valve. Kabbani proposes autoclaving the PET fabric in albumin for ten minutes to obtain the necessary stiffness. The use of a pulmonary valve autograft in the Ross II and Kabbani technique requires a lengthy surgical procedure, and requires additional surgery at the pulmonary valve site from which the autograft tissue is obtained. The technique also usually requires a homograft pulmonary valve, which is not easy to obtain, to replace the autograft.

CarboMedics manufactures a prosthetic mechanical aortic valve, also referred to as a “Top Hat,” in which the brim of the hat is seated on the downstream side of the aortic annulus, with a rigid tube extending downstream into the aortic root and supporting the valve mechanism. However, some surgeons, and some regulatory authorities, disfavor mechanical valves, because of a fear that they can encourage thrombus formation.

There is a need for a mitral valve prosthesis that can be installed in patients with significant calcification of the mitral annulus, and that avoids, or at least makes it possible to mitigate, some of the problems of the prior art.

### SUMMARY

In one embodiment, the present invention provides a valve comprising a frame defining cusps at a downstream end, between which extend valve leaflets arranged to allow fluid flow in a downstream direction but not in an upstream direction. A sewing ring is provided at the downstream end of the valve.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a side view of one form of valve according to an embodiment of the invention, for use as a mitral valve in a human being.

FIG. 2 is a perspective view of the frame and leaflets of the valve shown in FIG. 1.

### DETAILED DESCRIPTION

Reference will now be made in detail to various embodiments of the present invention, examples of which are illustrated in the accompanying drawings.

Referring to the drawings, one embodiment 20 of a valve according to the invention comprises a frame 22 comprising a sewing ring reinforcement 24, a valve support 26, and a top ring 28. The frame 22 is made of titanium, cobalt-nickel alloy, polytetrafluoroethylene, or other material that will not corrode in the environment of the human bloodstream and will not fatigue over the lifetime of the valve. The valve support 26 is generally in the form of three approximately semi-circular arcs 27, with the middle of each arc lying along and joined to the top ring 28, and the ends of adjacent arcs meeting in cusps 30 that are supported on the sewing ring reinforcement 24.

Three valve leaflets 32 of physiologically acceptable flexible material are attached to the valve support 26. In general, “physiologically acceptable” material may be a material that does not react unacceptably with blood or any constituent of blood, does not provoke an undue immune reaction, and does not tend to encourage thrombus formation. Physiologically acceptable materials may include autograft human tissue, homograft human tissue, heterograft tissue, and synthetic materials such as PET or polytetrafluoroethylene. Homograft and heterograft materials, in particular, may provoke some immune reaction that can be managed with immunosuppressant drugs. However, the use of synthetic materials that do not require the use of immunosuppression is presently preferred. Any thrombogenicity is preferably sufficiently slight that the long-term use of thrombolytic drugs is not necessary. The formation of a thin layer of new tissue over the synthetic material may be acceptable, and may even be encouraged, where the new tissue prevents further interaction between the patient's body and the synthetic material, provided that the new tissue does not hinder the operation of the valve.

Each valve leaflet 32 is attached along one of the arcs 27, with free edges 34 of the leaflet extending into the interior of the frame. The leaflets 32 are dimensioned so that under a liquid flow in the direction from the sewing ring reinforcement 24 to the top ring 28 the leaflets 32 spread towards one another, with the free edges meeting along radial commissures, closing off the space within the frame 22. Under a liquid flow in the opposite direction, the leaflets 32 fold apart, allowing liquid flow with little obstruction. The geometry of the leaflets 32 may be similar to that of conventional tri-leaflet heart valves that have been well known in the art for many years and, in the interests of conciseness, will not be further described here.

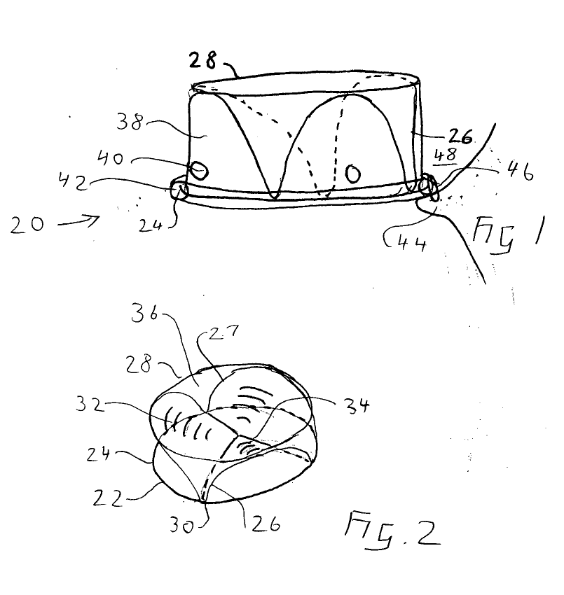

The spaces between the arcs 27 of the valve support 26 and the top ring 28 are filled with spandrels 36. A shroud 38 extends from the top ring 28 to the sewing ring reinforcement 24. The shroud 38 may be fastened to the arcs 27, in which case the upper part of the shroud 38 may form the spandrels 36. The spandrels 36 and the shroud 38 are made from physiologically acceptable material, for example, PET cloth. The shroud 38 is formed with one or more openings 40. The openings 40 are sufficiently few and small, relative to the open area within the valve 20, that back-flow through the openings 40 when the valve is closed does not render the valve more than trivially incompetent. As shown in FIG. 1, there is one opening 40 below each arc 27 of the frame 22. However, there may be more or fewer openings 40, for example, a single opening. The openings 40 may be circular, or of another suitable shape. The function of the opening 40 is explained in more detail below.

The sewing ring reinforcement 24 is covered in a sewing ring or cuff 42 of physiologically acceptable material. The sewing cuff 42 may be comparatively bulky and extend radially outward from the sewing ring reinforcement 24, to facilitate sewing the valve 20 to tissue radially outward of the cusps 30. Any parts of the frame 22 that are not otherwise covered are also covered by physiologically acceptable material to prevent interaction between the metal or other material of the frame and the patient's system.

In use, the valve 20 is placed within the left atrium, with the sewing cuff 42 resting on the mitral annulus and the valve extending upwards into the atrium. The sewing cuff 42 is sutured to the mitral annulus, or to adjacent tissue on the atrial side of the annulus, by sutures 46. The valve 20 may be selected so that the clear lumen within the rings 24, 28 is of approximately the same size as the mitral annulus. However, because the valve 20 does not need to fit within the mitral annulus, a slightly oversized valve can be used. As a result, the number of different sizes of valve 20 required to fit the normal range of human hearts may be reduced. For example, two sizes may be provided, as compared with the four sizes, in 2 mm increments, provided for most conventional mitral valve replacements.

Because the valve 20 is positioned entirely supra-annularly within the atrium, if the annulus is calcified the valve 20 may be selected so that the sutures 46 lie outside the area of calcification. However, it is usually desirable not to position the sutures 46 further outwards than is necessary. Therefore, the optimum size of the sewing cuff 42 may depend on the amount of calcification, which cannot always be determined accurately in advance. To accommodate this uncertainty, the valve 20 may be provided with an oversized, or maximally sized, sewing cuff 42 that can be cut down during surgery to the size actually required. The sewing cuff 42 may be wider on the posterior side, where calcification is more common.

If the native valve is stenotic, the native valve is at least partly excised or otherwise rendered at least partly incompetent to a sufficient extent that the native valve does not impede blood flow through the valve 20. If the native valve is incompetent, there is usually no reason to remove or alter the native valve and, to avoid unnecessary surgery, the native valve is usually allowed to remain in place.

Where, as is common, only the posterior mitral annulus is calcified, the sewing cuff 42 may be sewn to the uncalcified anterior mitral annulus, and sewn to the atrial wall above the calcified posterior mitral annulus. Alternatively, the sewing cuff 42 may be sutured to the native posterior mitral leaflet tissue below the region of calcification. This may result in a tilted position of the valve 20, but it is presently believed the tilting would not necessarily impair the functioning of the valve. Because the cusps 30 of the frame 22 are connected to the sewing ring reinforcement 24, and are thus close to the sutures 46 securing the valve in place, there is very little risk of the frame 22 becoming sufficiently displaced that the cusps 30 can cause damage to the surrounding heart tissue, even if the tips of the cusps 30 are not effectively shielded by being attached to the sewing ring reinforcement 24.

There is a region 48 surrounding the valve 20 that is outside the main flow of blood through the valve. In the arrangement shown in FIG. 1, the region 48 is open to the atrium. In order to reduce stagnation within the region 48, the openings 40 permit a limited reverse flow of blood from the ventricle to the atrium when the valve 20 is closed. The size of the openings 40 is limited so that the reverse flow is not sufficient to render the valve 20 functionally incompetent. The number of the openings 40 spaced around the circumference of the shroud 38 is sufficient to ensure backflow through substantially the whole circumference of the region 48. However, it is presently preferred to have as few openings 40 as is consistent with adequate flushing of the region 48. A single opening 40 may be sufficient. It is presently preferred to place the openings 40 close to the sewing cuff 42, in order to increase the proportion of the region 48 that is in the direct flow from the openings. It is preferred to form the openings 40 with smooth edges, to reduce the risk of hemolysis. Where the shroud 38 is made of fabric, a solid edge to the openings 40 may also reduce fraying of the fabric. Where the shroud 38 is a fabric of thermoplastic fibers, a suitably smooth, solid edge may be formable by fusing the thermoplastic.

Various modifications and variations can be made in the present invention without departing from the spirit or scope of the invention. For example, it may be possible to omit the top ring 28 and the spandrels 36, and extend the shroud 38 from the arcs 27 to the sewing ring reinforcement 24. However, the top ring 28 may be desirable to strengthen and stabilize the valve support 26. The top ring 28 and the valve support 26 may be combined into a single component. The parts of the valve support 26 between the cusps 30 may be of a shape other than the arcs 27. For example, the cusps 30 may be formed by posts extending approximately axially from the top ring 28, and the leaflets may then be attached to two posts and the section of the top ring 28 between the two posts, either directly or by way of spandrels 36.

The valve may have more or fewer than three leaflets 32, with the valve support 26 having correspondingly more or fewer arcs 27 and cusps 30.

For example, it may be possible to omit the sewing ring reinforcement 24 and connect the cusps 30 to a soft sewing cuff 42. When the valve 20 is placed supra-atrially as a mitral valve, the greatest force acting on the valve is an upward force during systole, placing the attachments between the frame 22 and the mitral annulus 46 in tension. This force can be transmitted by a flexible sewing cuff. However, some relatively rigid structure to distribute the loads on the valve 20 relatively uniformly around the mitral annulus may be desired.

For example, the sewing ring reinforcement 24 is shown in FIG. 1 as contacting the cusps 30 of the valve support 26. Especially where a relatively wide sewing cuff 42 is used to avoid calcification, the valve support 26 may be attached to the sewing ring reinforcement 24 by radial spacers. Alternatively, two reinforcing rings, spaced apart by radial spacers, may be used instead of the single sewing ring reinforcement shown in the drawings.

Specific materials and methods of construction have been shown and described. For example, the tri-leaflet valve of three leaflets 32 is presently preferred, because it has been used successfully for many years on many patients, and its design, construction, advantages and limitations are well understood. However, other designs of valve, including designs to be developed in the future, may be used, and may become preferred. For example, although a frame of metal wire or plastic has been described, new materials are being developed for bioprosthetic uses, and the frame may be formed from a material other than metal, including materials to be developed hereafter, and/or by a construction other than fabrication from wire.

Although the valve has been described with reference to a mitral valve for a human heart, the valve may have other uses, for example, as a tricuspid or other valve, as a valve for non-human hearts, or as a valve for purposes other than as a heart valve.

Thus, it is intended that the present invention cover the modifications and variations of this invention provided they come within the scope of the appended claims and their equivalents.

What is claimed is:

1. A valve comprising:
a frame defining cusps at a downstream end;
valve leaflets attached to the frame arranged to allow fluid flow within the frame in a downstream direction but not in an upstream direction;
a sewing ring at the downstream end of the valve; and
a shroud connecting the sewing ring to the frame.

2. The valve according to claim 1, wherein the sewing ring comprises a reinforcing ring fixed to the cusps of the frame.

3. The valve according to claim 1, wherein the valve leaflets are attached along sections of the frame extending from one cusp to the next upstream of the cusps.

4. The valve according to claim 3, wherein the sections of the frame upstream of the cusps comprise arcs extending from cusp to cusp.

5. The valve according to claim 4, wherein the shroud comprises spandrel sections between adjacent arcs on the side away from the sewing ring.

6. The valve according to claim 5, wherein the frame comprises a reinforcing ring at the upstream end, and the spandrel sections extend from the arcs to the reinforcing ring.

7. The valve according to claim 1, wherein the frame comprises a reinforcing ring at the upstream end, and the shroud comprises a shroud section extending from the reinforcing ring to the sewing ring.

8. The valve according to claim 1, wherein the shroud is provided with one or more apertures permitting limited upstream flow.

9. The valve according to claim 1, wherein the sewing ring is spaced outward from the cusps.

10. The valve according to claim 1, suitable for use as a replacement heart valve.

11. The valve according to claim 10, suitable for use as a mitral valve replacement in a human heart.
